(19) United States
(12) Patent Application Publication
(10) Pub. No.: US 2014/0369998 A1
(43) Pub. Date: Dec. 18, 2014

(54) Biomarkers for Tuberculosis and HIV/AIDS

(71) Applicants: Andries J.C. Steyn, Hoover, AL (US); Michael Kimerling, Seattle, WA (US); German Henostroza, Birmingham, AL (US); David K. Crossman, Hoover, AL (US)

(72) Inventors: Andries J.C. Steyn, Hoover, AL (US); Michael Kimerling, Seattle, WA (US); German Henostroza, Birmingham, AL (US); David K. Crossman, Hoover, AL (US)

(21) Appl. No.: 13/992,458

(22) PCT Filed: Dec. 9, 2011
(86) PCT No.: PCT/US11/64194

§ 371 Date: Aug. 12, 2014

Related U.S. Application Data
(60) Provisional application No. 61/421,482, filed on Dec. 9, 2010.

(51) Int. Cl. G01N 33/569; G01N 33/68

(52) U.S. Cl. CPC G01N 33/5695; G01N 33/56988; G01N 33/6863; G01N 33/6869; G01N 2333/523; G01N 2333/525; G01N 2333/535; G01N 2333/55; G01N 2333/5412; G01N 2333/5418; G01N 2333/5415; G01N 2333/5434; G01N 2333/70503; G01N 2333/70546; G01N 2800/60; G06F 19/10
USPC 424/130.1; 506/9; 702/19

(57) ABSTRACT

Described herein is a diagnostic test that identifies circulating biomarkers for the differentiation and classification of the pathogenesis of TB and/or HIV in a population.

### CROSS-REFERENCE TO PRIORITY APPLICATION

This application claims priority to U.S. Provisional Application No. 61/421,482, filed Dec. 9, 2010, which is incorporated herein by reference in its entirety.

### BACKGROUND

Diagnosing tuberculosis (TB) is a cumbersome task in countries where TB is endemic. The primary diagnostic test is obtaining sputum samples from patients and examining for acid-fast bacilli by microscopy. Multiple specimens and visits of the patient are required and this significantly increases the drop-out rate of patients who might be infected and thus leading to untreated TB. Unfortunately, the TB epidemic is not under control, making Mycobacterium tuberculosis (Mtb) the causative agent of TB a major health problem in where one-third of the world's population is latently infected. One out of every 10 infected TB patients will actually develop this disease, but this percentage increases significantly in those who are coinfected with both Mtb and HIV. Fortunately, TB, if caught early, can be treated, leading to fewer deaths. The drug regime is long and tedious consisting of 3 to 4 drugs over a period of 6 to 9 months, which leads to poor compliance and is the main cause of the emergence of single drug-resistant, multidrug resistant (MDR) and extensively drug-resistant (XDR) strains of Mtb.

### SUMMARY

Provided herein are methods for determining the HIV status, the TB status and/or the purified protein derivative (PPD) status of a subject by measuring cytokine levels and utilizing predictive equations. Further provided are methods of treating HIV infection and/or TB infection.

### BRIEF DESCRIPTION OF THE DRAWINGS

FIGS. 1A-C show HIV frequency plots of cytokines (PDGF ββ, SCF and eotaxin).

FIGS. 2A-D show TB frequency plots of cytokines (GRO-α, MCP-3, TNF-β and MCSF).

FIGS. 3A-D show PPD+ vs. all others frequency plots of cytokines (MCP-3, LIF, CTACK and ICAM).

FIG. 4 shows PPD+ vs. TB− Frequency plots of cytokines (ICAM-1).

FIG. 5 shows PPD+ vs. healthy frequency plots of cytokines (ICAM-1).

### DETAILED DESCRIPTION

The present method provides for determining the HIV status, the TB status and/or the purified protein derivative (PPD) status of a subject by measuring cytokine levels and utilizing predictive equations. More specifically, the method includes diagnosing a subject as HIV+ or HIV− comprising (a) measuring the levels of eotaxin, stem cell factor (SCF), and platelet derived growth factor bb (PDGF ββ) in a biological sample from the subject and (b) computing a predictive value utilizing the following equation:

$$p = \frac{1}{1 + {e}^{- z}}$$

where z=5.654+0.003*Eotaxin−0.032*SCF−0.001*PDGF ββ,
wherein a value >0.5 predicts HIV+, and a value <0.5 predicts HIV−, thus diagnosing the subject as HIV+ or HIV−. Optionally, the method further comprises taking steps to initiate or alter treatment of the subject based on the determination.

As used herein, a biological sample is a sample derived from a subject and includes, but is not limited to, any cell, tissue or biological fluid. The sample can be, but is not limited to, peripheral blood, plasma, urine, saliva, gastric secretion or bone marrow specimens.

As used throughout, by subject is meant an individual. Preferably, the subject is a mammal such as a primate, and, more preferably, a human. Non-human primates include marmosets, monkeys, chimpanzees, gorillas, orangutans, and gibbons, to name a few. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Veterinary uses and formulations for same are also contemplated herein.

Also provided is a method of diagnosing a subject as TB+ or TB− comprising (a) measuring the levels of macrophage colony stimulating factor (MCSF), tumor necrosis factor beta (TNFBeta), monocyte chemoattractant protein 3 (MCP3), and melanoma growth stimulating activity, alpha (GROalpha) in a sample from a subject and (b) computing a predictive value utilizing the following equation:

$$p = \frac{1}{1 + {e}^{- z}}$$

where z=2.146+0.066*MCSF+0.593*TNFβ−0.058*MCP3+0.012GROα, wherein a value >0.5 predicts TB+, and a value <0.5 predicts TB−, thus diagnosing the subject as TB+ or TB−. Optionally, the method further comprising taking steps to initiate or alter treatment of the subject based on the determination.

Also provided is a method of diagnosing a subject as PPD+ or not PPD+ comprising a) measuring the levels of leukemia inhibitory factor (LIF), MCP3, chemokine (C—C motif) ligand 27 (CTACK) and intercellular adhesion molecule 1 (ICAM-1) in a sample from a subject and b) computing a predictive value utilizing the following equation:

$$p = \frac{1}{1 + {e}^{- z}}$$

where z=−0.611−0.055*LIF+0.009*MCP3+0.001*CTACK−0.141*ICAM1/1000 wherein a value >0.5 predicts PPD+, and a value <0.5 predicts not PPD+, thus diagnosing the subject as PPD+ or not PPD+. Optionally, the method further comprising taking steps to initiate or alter treatment of the subject based on the determination.

Also provided is a method of diagnosing a subject that is TB− as PPD+ or not PPD+ comprising a) measuring the levels of ICAM in a sample from a TB− subject and b) computing a predictive value utilizing the following equation:

$$p = \frac{1}{1 + {e}^{- z}}$$

where z=14.508−0.549*ICAM1/1000
wherein a value >0.5 predicts PPD+, and a value of <0.5 predicts not PPD+.

Table 1 sets forth identifying information for the proteins utilized in the predictive equations provided herein. Column 1 of Table 1 provides the name of the protein. Column 2 of Table 1 provides one or more aliases for each of the proteins. Therefore, it is clear that when referring to a protein, this also includes known alias(es) and any aliases attributed to the proteins listed in Table 1 in the future. Also provided in Table 1 are the GenBank Accession Nos. for the coding sequences (human mRNA sequences) (column 6) and the GenBank Accession Nos. for the human protein sequences (column 7). The nucleic acid sequences and protein sequences provided under the GenBank Accession numbers mentioned herein are hereby incorporated in their entireties by this reference. One of skill in the art would know that the nucleotide sequences provided under the GenBank Accession numbers set forth herein can be readily obtained from the National Center for Biotechnology Information at the National Library of Medicine (http://www.ncbi.nlm.nih.gov/entrez/query.fcgi?db=nucleotide). Similarly, the protein sequences set forth herein can be readily obtained from the National Center for Biotechnology Information at the National Library of Medicine (http://www.ncbi.nlm.nih.gov/entrez/query.fcgi?db=protein). The nucleic acid sequences and protein sequences provided under the GenBank Accession numbers mentioned herein are hereby incorporated in their entireties by this reference. Further provided are the Entrez Gene numbers for the human genes (column 8). The information provided under the Entrez Gene numbers listed in Table 1 is also hereby incorporated entirely by this reference. One of skill in the art can readily obtain this information from the National Center for Biotechnology Information at the National Library of Medicine (http://www.ncbi.nlm.nih.gov/entrez/query.fcgi?db=gene).

These examples are not meant to be limiting as one of skill in the art would know how to obtain additional sequences for proteins and nucleic acids encoding the proteins listed in Table 1 from other species by accessing GenBank (Benson et al. Nucleic Acids Res. 2004 Jan. 1; 32(Database issue); D23-D26), the EMBL Database (Stoesser et al., (2000) Nucleic Acids Res., 28, 19-23) or other sequence databases. One of skill in the art would also know how to align the sequences disclosed herein with sequences from other species in order to determine similarities and differences between the sequences set forth in Table 1 and related sequences, for example, by utilizing BLAST.

TABLE 1

| | | | Human | | |
|---|---|---|---|---|---|
| | | | GenBank | | |
| | | | Accession No. | Human | |
| | | | for coding | GenBank | Entrez |
| | | | sequence/ | Accession No. | Gene |
| Protein | Alias | Definition | mRNA | for protein | No. |
| eotaxin | CCL11, | Eosinophil | NM_002986.2 | NP_002977.1 | 6356 |
| | SCYA11 | chemotactic protein | | | |
| SCF | SF, MGF, | Stem cell factor; kit | NM_000899.4 | NP_000890.1 | 4254 |
| | FPH2, KL- | ligand | NM_003994.5 | NP_003985.2 | |
| | 1, Kit1; | | | | |
| | SHEP7; | | | | |
| | kit-ligand | | | | |
| PDGF ββ | PDGF2, | PDGF-BB | NM_002608.2 | NP_002599.1 | 5155 |
| Homodimer | SIS, SSV, | (homodimer of | NM_033016.2 | NP_148937.1 | |
| of PDGF | c-sis | PDGF-B) | | | |
| subunit b | | | | | |
| MCSF | CSF-1 | Colony stimulating | NM_000757.5 | NP_000748.3 | 1435 |
| | | factor 1 | NM_172210.2 | NP_757349.1 | |
| | | (macrophage) | NM_172212.2 | NP_757351.1 | |
| TNFβ | TNFB, | Tumor necrosis | NM_000595.2 | NP_000586.2 | 4049 |
| | TNFSF1 | factor beta | NM_001159740.1 | NP_001153212.1 | |
| MCP3 | FIC, | Monocyte | NM_006273.2 | NP_006264.2 | 6354 |
| | MARC; | chemotactic protein- | | | |
| | NC28; | 3 | | | |
| | MCP-3; | | | | |
| | SCYA6; | | | | |
| | SCYA7 | | | | |
| GROα | FSP, | Melanoma growth | NM_001511.2 | NP_001502.1 | 2919 |
| | GRO1, | stimulating activity, | | | |
| | GROa, | alpha | | | |
| | MGSA; | | | | |
| | NAP-3, | | | | |
| | SCYB1, | | | | |
| | MGSA-a | | | | |
| LIF | CDF, DIA, | Leukemia inhibitory | NM_002309.3 | NP_002300.1 | 3976 |
| | HILDA | factor | | | |
| CTACK | ALP, ILC, | Chemokine (C-C | NM_006664.2 | NP_006655.1 | 10850 |
| | CTAK, | motif) ligand 27 | | | |
| | PESKY, | | | | |
| | ESKINE, | | | | |
| | SCYA27 | | | | |
| ICAM | BB2, | Intracellular | NM_000201.2 | NP_000192.2 | 3383 |
| | CD54, | adhesion molecule 1 | | | |
| | P3.58 | | | | |

In the present methods, the levels of cytokines can be measured in picograms per milliliter (pg/ml) or micrograms per deciliter (μg/dl), for example. Protein levels or concentration can be determined by methods standard in the art for quantitating proteins, such as Western blotting, ELISA, ELISPOT, immunoprecipitation, immunofluorescence (e.g., FACS), immunohistochemistry, immunocytochemistry, etc., as well as any other method now known or later developed for quantitating protein in or produced by a cell.

As utilized herein PPD means Purified protein derivative (PPD) tuberculin, TB means tuberculosis and HIV means human immunodeficiency virus. In the methods described herein, measuring the levels of the cytokines in the subject can be but is not necessarily performed by the individual that obtains the sample or the individual that computes the predictive values from the equations set forth herein. Also provided herein are methods of obtaining levels of cytokines in a sample from a subject in the form of numerical data, for example, via any means of data transmission, such as from a database, a laboratory report, a CD-ROM, electronic mail, etc. and entering the values into the predictive equations to obtain the HIV, TB and/or PPD status of the subject.

The methods set forth herein can be utilized to diagnose a subject as HIV+, TB+, HIV+/TB+, HIV−/TB+, HIV−TB−, HIV+/TB−/PPD+, HIV+/TB−/PPD−, HIV−/TB−/PPD+, or HIV−/TB−/PPD−. For example, and not to be limiting, levels of cytokines in the predictive HIV equation (eotaxin, SCF, PDFG ββ) and/or levels of cytokines in the predictive TB equation (MCSF, TNFBeta, MCP3, GROalpha) can be measured in a sample from a subject to determine the HIV and/or TB status of the subject. In addition, the levels of the cytokines in the predictive PDD equations (LIF, MCP3, CTACK and ICAM-1) can be measured in a sample from a subject to determine the PPD status of the subject.

Once a diagnosis is made, for example, HIV+/TB+, the appropriate composition, for example, drug(s) or other therapy(ies) can be selected and administered for treatment of the coinfected subject. The composition can comprise, for example, a chemical, a compound, a small molecule, an aptamer, a drug, a protein, a cDNA, an antibody, a morpholino, a triple helix molecule, an siRNA, an shRNAs, an antisense nucleic acid or a ribozyme.

Compounds that decrease HIV infection and/or compounds that decrease tuberculosis infection can be utilized. Antiviral compounds useful in the treatment of HIV include, but are not limited to Combivir® (lamivudine-zidovudine), Crixivan® (indinavir), Emtriva® (emtricitabine), Epivir® (lamivudine), Fortovase® (saquinavir-sg), Hivid® (zalcitabine), Invirase® (saquinavir-hg), Kaletra® (lopinavir-ritonavir), Lexiva™ (fosamprenavir), Norvir® (ritonavir), Retrovir® (zidovudine), Sustiva® (efavirenz), Videx EC® (didanosine), Videx® (didanosine), Viracept® (nelfinavir) Viramune® (nevirapine), Zerit® (stavudine), Ziagen® (abacavir), Fuzeon® (enfuvirtide) Rescriptor® (delavirdine), Reyataz® (atazanavir), Trizivir® (abacavir-lamivudine-zidovudine) Viread® (tenofovir disoproxil fumarate), Agenerase® (amprenavir) and combinations thereof. Compounds that can be used to treat tuberculosis include, but are not limited to, ethambutol, isoniazid, pyrazinamide, rifampicin, amikacin, kanamycin, capreomycin, viomycin, enviomycin, fluoroquinones (for example, ciprofloxacin, levofloxoacin and moxifloxacin), ethionamide, prothionamide, rifabutin, clarithromycin, linezoid, thioacetazone, thioridazine, arginine, vitamin D, R207910 and combinations thereof. Any combination of a compound(s) utilized to treat HIV and a compound(s) utilized to treat tuberculosis can be utilized to treat a subject coinfected with tuberculosis and HIV. Similarly, if the patient is HIV+/TB−, the appropriate drug(s) or other therapy(ies) to treat only HIV can be administered. Further, if the patient is HIV−/TB+, the appropriate drug(s) or other therapy(ies) to treat only tuberculosis can be administered.

Depending on the intended mode of administration, the composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the compounds described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are preferably suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the compounds described herein or derivatives thereof include ointments, powders, sprays, gels and the like. The compounds described herein or derivatives thereof are admixed under sterile conditions with a physiologically acceptable carrier and any preservatives, buffers, or propellants as may be required.

Throughout this application, by treat, treating, or treatment is meant a method of reducing the effects of an existing infection. Treatment can also refer to a method of reducing the disease or condition itself rather than just the symptoms. The treatment can be any reduction from native levels and can be, but is not limited to, the complete ablation of the disease or the symptoms of the disease. Treatment can range from a positive change in a symptom or symptoms of infection to complete amelioration of the an infection as detected by art-known techniques. For example, a disclosed method is considered to be a treatment if there is about a 10% reduction in one or more symptoms of the disease in a subject with the disease when compared to native levels in the same subject or control subjects. Thus, the reduction can be about a 10, 20, 30, 40, 50, 60, 70, 80, 90, 100%, or any amount of reduction in between as compared to native or control levels.

As utilized herein, by prevent, preventing, or prevention is meant a method of precluding, delaying, averting, obviating, forestalling, stopping, or hindering the onset, incidence, severity, or recurrence of infection. For example, if a subject is found to be HIV+/TB−/PPD+, antimicrobial therapy can be administered prophylactically to prevent tuberculosis infection.

Administration can be carried out using therapeutically effective amounts of the agents described herein for periods of time effective to treat or prevent infection. The effective amount may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day.

The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration may be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment, for example by topical application or local injection. Multiple administrations and/or dosages can also be used. Effective doses can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the following claims. The following examples are exemplary of the invention and are not intended to limit the scope of what the inventors regard as their invention.

### Examples

Described herein is a diagnostic test that identifies circulating biomarkers for the differentiation and classification of the pathogenesis of TB and/or HIV in a population. Currently, there are few, if any studies predicting biomarkers for both TB and HIV populations. Instead, the focus has been on predicting biomarkers for only TB or HIV populations. This is the first large scale study of circulating cytokines, chemokines and growth factors in a clinically relevant population. Out of the 50 cytokines, chemokines and growth factors tested, several were found to be candidates for new diagnostic tests to validate novel drug and vaccine candidates, and to identify patients with TB and/or HIV in which a diagnosis can be pronounced within days, and the appropriate drug regimen prescribed.

Mycobacterium tuberculosis (Mtb), the causative agent of tuberculosis (TB) is a major health problem and it is estimated that one-third of the world's population is latently infected. Typically, only 1 in 10 will develop the disease, but this percentage increases dramatically in those who are coinfected with Mtb and HIV. Most deaths from TB are avoidable by early diagnosis and treatment. However, more than 10% of HIVfTB-coinfected persons may have a negative tuberculin skin test as a result of anergy. Here, we examined the profiles of 50 cytokines, chemokines and growth factors in the sera of 207 PPD−/HIV− (healthy controls), PPD+/HIV− (latent TB), HIV−/TB+(active disease), HIV+/TB+ coinfected, and HIV+/PPD-patients from Peru. After estimating the univariate statistics for the cytokine intensity in each group, Analysis of Variance (ANOVA) was used to test for differences across groups. Once statistically significant differences between the groups were identified, Principal Component Analysis (PCA) was used to examine the ability of the cytokines to cluster the disease groups. A quadratic discriminant analysis procedure was used to test the capacity of the cytokines to discriminate between the five groups. Leave-One-Out-Cross-Validation (LOOCV) was used to examine the quality of the discrimination. The data showed that several cytokines, chemokines and growth factors tested were able to classify disease, or disease state. The biomarkers identified in this study are candidates that could be used to develop new TB and/or TB/HIV diagnostic tests.

Sera was collected in a blinded fashion from 207 patients from Peru and stored at −80° C. until tested in a blinded fashion by using Bio-Rad's multiplex bead array approach based from the Luminex technology. After analysis the samples were un-blinded and categorized into their appropriate groups. Of these 207 patients, 34 were PPD−/HIV (healthy controls) containing 15 males and 19 females aged 22 to 49, 44 were PPD+/HIV (latent TB) containing 21 males and 23 females aged 20 to 61, 55 were HIV−ITB+(active disease) containing 27 males and 28 females aged 19 to 61, 58 were HIV+/TB+(coinfected) containing 28 males and 30 females aged 22 to 55, and 16 were HIV+/PPD− containing 11 males and 5 females aged 18 to 49.

### Cytokine Analysis

The Bio-Rad Bio-Plex Human Cytokine 27-Plex Panel (Catalog #171-A11127) and Human Cytokine 23-Plex Panel (Catalog #171-A11123) (Bio-Rad, CA) were performed on the Peru samples in triplicate according to the manufacturer's instructions. The 50 cytokines, chemokines and growth factors analyzed were IFN-α2, IL-1a, IL-1(3, IL-1ra, IL-2, IL-2ra, IL-3, IL-4, IL-5, IL-6, IL-7, IL-8, IL-9, IL-10, IL-12 (p40), IL-12 (p70), IL-13, IL-15, IL-16, IL-17, IL-18, CTACK, Eotaxin, FGFbasic, G-CSF, GM-CSF, GRO-a, HGF, ICAM-1, IFN-y, IP-10, LIF, MCP-1 (MCAF), MCP-3, M-CSF, MIF, MIG, MIP-1a, MIP-1I3, B-NGF, PDGF bb, RANTES, SCF, SCGF-I3, SDF-1a, TNF-a, TNF-I3, TRAIL, VCAM-1 and VEGF. Table 2 provides the results of the analysis.

TABLE 2
Cytokine values were compared between the five groups using a Kruskal-Wallis test
(non-parametric ANOVA). Data are given as median (minimum, maximum) since the values
are not normally distributed and means and standard deviations would not be appropriate.

| | Healthy | | Latent | | TB | |
|---|---|---|---|---|---|---|
| | (HIV⁻ TB⁻) | | (HIV⁻ PPD⁺ TB⁻) | | (HIV⁻ TB⁺) | |
| | Median | Range | Median | Range | Median | Range |
| IL-1β | 4.8 | 3.3-44.0 | 5.4 | 3.9-9.4 | 5.4 | 3.1-70.0 |
| IL-1ra | 222.7 | 115.0-1956 | 230.5 | 95.5-843.6 | 262.9 | 139.8-10944 |
| IL-2 | 5.5 | 0.1-135.1 | 6.2 | 0.1-57.3 | 6.7 | 0.1-1019 |
| IL-4 | 26.5 | 18.1-49.9 | 24.2 | 13.7-37.3 | 23.2 | 13.7-77.4 |
| IL-5 | 1.5 | 0.2-91.8 | 1 | 0.3-5.7 | 1.1 | 0.1-9.3 |
| IL-6 | 11.3 | 5.4-143.4 | 17.9 | 7.1-162.8 | 35.6 | 7.1-638.9 |
| IL-7 | 8.4 | 3.9-457.1 | 12 | 2.9-34.1 | 17 | 2.8-40.6 |
| IL-8 | 12.3 | 4.4-558.7 | 11.1 | 3.9-2340 | 25.5 | 3.8-31593 |
| IL-9 | 45.6 | 15.2-331.9 | 53.7 | 5.7-322.5 | 59 | 21.5-13845 |
| IL-10 | 6.6 | 1.0-1699 | 8.6 | 1.4-31.1 | 10.2 | 2.0-46.1 |
| IL-12 (p70) | 6.6 | 2.2-602.0 | 7.3 | 2.1-23.5 | 8.2 | 2.7-66.6 |
| IL-13 | 20.6 | 5.9-1872 | 27.1 | 8.0-69.8 | 29.1 | 11.5-88.9 |
| IL-15 | 8.9 | 0.1-64.5 | 7.7 | 0.1-25.2 | 9.8 | 0.1-490.5 |
| IL-17 | 6.9 | 0.1-49.3 | 6.5 | 0.1-34.8 | 9.9 | 0.1-77.4 |
| Eotaxin | 95.3 | 2.2-853.6 | 111.1 | 13.4-791.3 | 78.9 | 13.4-2780 |
| FGF basic | 33.6 | 1.0-96.4 | 32.1 | 1.0-189.4 | 40.3 | 1.0-189.4 |
| G-CSF | 33.3 | 20.7-88.6 | 30.1 | 19.7-51.5 | 36.1 | 21.7-145.50 |
| GM-CSF | 21.9 | 0-138.1 | 16.8 | 0-67.7 | 9 | 0-466.8 |
| IFN-γ | 203.3 | 137.6-394.7 | 187.8 | 108.5-445.1 | 171.4 | 111.2-912.1 |
| IP-10 | 1189 | 235.2-7129 | 1158 | 153.8-5708 | 1948 | 203.2-50939 |
| MCP-1 | 3.2 | 0.1-237.9 | 0.9 | 0.1-76.0 | 0.1 | 0.1-76.0 |
| MIP-1α | 20.3 | 13.8-295.2 | 18.6 | 13.5-191.2 | 21.5 | 13.5-1408 |
| PDGF bb | 7635 | 2359-16768 | 6817 | 1735-21149 | 5752 | 1735-19323 |
| TNF-α | 23.1 | 1.3-94.1 | 24.1 | 1.3-188.5 | 19.6 | 1.3-354.2 |
| VEGF | 125.4 | 1.7-656.5 | 200.7 | 7.0-1387 | 282.3 | 3.7-1387 |
| IFN-α2 | 206.4 | 140.2-366.7 | 207.1 | 128.8-450.0 | 227.1 | 135.3-404.9 |
| IL1-α | 0.005 | 0.005-2.51 | 0.005 | 0.005-3.63 | 0.89 | 0.005-7.38 |
| IL-2rα | 282.5 | 128.2-628.7 | 243.1 | 98.4-620.2 | 347.8 | 129.8-986.8 |
| IL-3 | 143.3 | 2.1-462.5 | 96.1 | 2.1-485.5 | 149.3 | 2.1-384.7 |
| IL-12 (p40) | 1043 | 223.2-2968 | 773.2 | 202.0-2534 | 1117 | 175.0-2540 |
| IL-16 | 193.5 | 95.4-456.7 | 197.8 | 89.4-576.7 | 263.4 | 112.6-526.5 |
| IL-18 | 157.7 | 68.1-363.5 | 119.7 | 40.9-407.3 | 252.6 | 83.9-1438 |
| CTACK | 1079 | 496.7-1983 | 1410 | 704.7-2038 | 1385 | 483.1-2302 |
| GRO-α | 153.4 | 4.3-370.3 | 171.2 | 4.3-440.5 | 348.3 | 116.4-1288 |
| HGF | 821.7 | 267.4-1439 | 938.5 | 405.8-1646 | 1416 | 513.4-4883 |
| ICAM-1 | 30083 | 21596-30083 | 23009 | 19587-30083 | 30083 | 22208-30083 |
| LIF | 0.005 | 0.005-79.9 | 0.005 | 0.005-64.2 | 13.4 | 0.005-72.4 |
| MCP-3 | 149.7 | 37.0-329.9 | 197.4 | 87.3-479.3 | 108.1 | 33.7-409.2 |
| M-CSF | 30.1 | 2.3-106.8 | 39.4 | 2.3-183.7 | 83.5 | 17.6-195.2 |
| MIF | 301.8 | 122.5-31600 | 334.9 | 138.2-3153 | 1191 | 199.1-31600 |
| MIG | 1976 | 355.8-20401 | 1782 | 316.0-35285 | 10979 | 894.6-35285 |
| β-NGF | 5.5 | 2.4-9.2 | 5.5 | 3.0-13.6 | 5.8 | 2.3-10.4 |
| SCF | 114.7 | 54.4-211.3 | 102.1 | 34.7-179.8 | 109 | 46.8-195.8 |
| SCGF-β | 99,291 | 6277-251226 | 122,270 | 31672-251226 | 103,675 | 2184-230161 |
| SDF-1α | 1388 | 741.1-2749 | 1388 | 681.9-3744 | 1395 | 630.0-3168 |
| TNF-β | 0.005 | 0.005-9.7 | 0.005 | 0.005-36.5 | 6.9 | 0.005-51.3 |
| TRAIL | 334.3 | 128.3-714.8 | 283.5 | 83.9-973.5 | 335.5 | 56.5-778.4 |
| VCAM-1 | 26905 | 19221-26905 | 21245 | 9683-26903 | 26905 | 20284-26905 |

| | HIV/TB | | HIV | | |
|---|---|---|---|---|---|
| | (HIV⁺ TB⁺) | | (HIV⁺ TB⁻) | | |
| | Median | Range | Median | Range | K-W Test |
| IL-1β | 5.8 | 3.1-663.7 | 3.5 | 1.5-101.8 | p < 0.001 |
| IL-1ra | 210.7 | 62.9-40807 | 156.6 | 18.4-6303 | p = 0.002 |
| IL-2 | 1.9 | 0.1-10370 | 0.1 | 0.1-423.9 | p < 0.001 |
| IL-4 | 27.5 | 9.7-878.3 | 15.7 | 4.8-154.0 | p = 0.001 |
| IL-5 | 3.3 | 0.8-68.1 | 1.8 | 0.2-44.0 | p < 0.001 |
| IL-6 | 14.7 | 2.9-2084 | 13.4 | 0.7-429.5 | p < 0.001 |
| IL-7 | 7.2 | 1.2-205.8 | 7.9 | 4.4-22.3 | p < 0.001 |
| IL-8 | 3.3 | 0.1-1230 | 5.3 | 0.2-31.0 | p < 0.001 |
| IL-9 | 76.2 | 13.0-1050 | 111.3 | 22.0-629.3 | p = 0.002 |
| IL-10 | 2.5 | 0.2-2348 | 4.1 | 0.2-168.8 | p < 0.001 |
| IL-12 (p70) | 5.7 | 0.01-2859 | 4.65 | 1.0-77.6 | p = 0.004 |
| IL-13 | 24.7 | 1.0-1702 | 37.1 | 3.4-343.7 | p = 0.127 |
| IL-15 | 1.5 | 0.1-81.8 | 4.8 | 0.1-213.3 | p < 0.001 |
| IL-17 | 0.1 | 0.1-42.9 | 0.1 | 0.1-20.0 | p < 0.001 |
| Eotaxin | 38.8 | 2.2-591.8 | 2.2 | 2.2-2291 | p < 0.001 |
| FGF basic | 35.7 | 1.0-162.9 | 39 | 1.0-94.9 | p = 0.809 |
| G-CSF | 38.9 | 20.9-2637 | 28.4 | 18.4-233.1 | p < 0.001 |
| GM-CSF | 25.6 | 0-28166 | 6.1 | 0-2204 | p = 0.008 |
| IFN-γ | 203.2 | 90.2-20555 | 143 | 57.9-2385 | p < 0.001 |
| IP-10 | 1636 | 389.5-50939 | 2202 | 638.2-22808 | p = 0.027 |
| MCP-1 | 0.1 | 0.1-556.4 | 0.1 | 0.1-139.8 | p < 0.001 |
| MIP-1α | 27.6 | 14.5-43.0 | 21 | 13.7-33.0 | p < 0.001 |
| PDGF bb | 616.2 | 107.7-8489 | 245.9 | 57.1-11263 | p < 0.001 |
| TNF-α | 29.5 | 1.3-6126 | 6.8 | 1.3-2266 | p = 0.005 |
| VEGF | 0.1 | 0.1-343.0 | 33.6 | 0.1-312.0 | p < 0.001 |
| IFN-α2 | 196.5 | 135.8-543.2 | 230.4 | 187.5-284.2 | p = 0.076 |
| IL1-α | 0.2 | 0.005-7.35 | 0.005 | 0.005-0.33 | p < 0.001 |
| IL-2rα | 268.1 | 100.1-1334 | 418.8 | 232.1-1398 | p < 0.001 |
| IL-3 | 97.3 | 2.1-1191 | 137.7 | 23.5-1866 | p = 0.019 |
| IL-12 (p40) | 703.5 | 75.4-2560 | 1217 | 614.8-5284 | p = 0.003 |
| IL-16 | 387 | 112.5-2433 | 439.8 | 155.7-1322 | p < 0.001 |
| IL-18 | 141.5 | 25.3-1077 | 189.9 | 73.6-428.6 | p < 0.001 |
| CTACK | 1143 | 575.5-2422 | 1200 | 814.9-1860 | p = 0.005 |
| GRO-α | 193.9 | 47.8-428.5 | 170.3 | 4.3-278.7 | p < 0.001 |
| HGF | 516 | 270.7-1221 | 584 | 371.4-2124 | p < 0.001 |
| ICAM-1 | 30083 | 8021-30083 | 30083 | 30083-30083 | p < 0.001 |
| LIF | 3.46 | 0.005-58.0 | 0.4 | 0.005-28.6 | p < 0.001 |
| MCP-3 | 94 | 39.4-349.8 | 131.2 | 62.1-857.9 | p < 0.001 |
| M-CSF | 48.7 | 13.1-190.5 | 26.2 | 4.9-59.9 | p < 0.001 |
| MIF | 1358 | 258.5-16105 | 1245 | 231.0-5110 | p < 0.001 |
| MIG | 1904 | 447.9-18522 | 4206 | 716.8-20205 | p < 0.001 |
| β-NGF | 6 | 2.6-13.6 | 6.4 | 3.2-9.2 | p = 0.634 |
| SCF | 83.6 | 35.5-153.6 | 86.8 | 50.3-265.4 | p < 0.001 |
| SCGF-β | 36,088 | 3852-251226 | 90,093 | 18417-251226 | p < 0.001 |
| SDF-1α | 1051 | 457.4-1985 | 1398 | 984.3-4004 | p < 0.001 |
| TNF-β | 2.48 | 0.005-34.6 | 0.005 | 0.005-2.6 | p < 0.001 |
| TRAIL | 290.4 | 65.0-694.4 | 425.2 | 219.2-1375 | p = 0.101 |
| VCAM-1 | 26905 | 7629-26905 | 26905 | 26905-26905 | p < 0.001 |

### HIV Logistic Regression

| Cytokine | Odds Ratio | 95% C.I. | p value | OR for δ =100 |
|---|---|---|---|---|
| GRO-α | 0.996 | 0.994, 0.999 | 0.003 | 0.670 |
| IL2 | 1.000 | 0.999, 1.001 | 0.422 | |
| IL17 | 0.811 | 0.748, 0.880 | <0.001 | 0.124* |
| SCF | 0.977 | 0.967, 0.987 | <0.001 | 0.091 |
| IL12 p70 | 1.001 | 0.999, 1.003 | 0.373 | |
| PDGF ββ | 0.999 | 0.999, 0.999 | <0.001 | 0.905 |
| Eotaxin | 0.997 | 0.995, 1.000 | 0.045 | 0.741 |
| IL4 | 1.008 | 0.993, 1.022 | 0.296 | |
| SDF-1α | 0.999 | 0.998, 0.999 | <0.001 | 0.905 |

Of the 9 cytokines that looked promising in the first step, six of them are significantly predictive of the presence of HIV using logistic regression. Since HIV is coded as 0=no and 1=yes, Odds Ratios of <1 imply that lower values are associated with presence of HIV. An Odds Ratio of >1 would imply that higher values are associated with the presence of HIV. The six significant cytokines were entered into a forward stepwise regression using the Likelihood ratio test to determine which variables entered the equation.

### Multivariable Logistic Regression

| Cytokine | Odds Ratio | 95% C.I. | p value | OR for δ = 100 |
|---|---|---|---|---|
| PDGF ββ | 0.999 | 0.999, 0.999 | <0.001 | 0.905 |
| SCF | 0.969 | 0.947, 0.991 | 0.005 | 0.041 |
| Eotaxin | 1.003 | 1.000, 1.005 | 0.048 | 1.350 |

### HIV Logistic Regression

The effect of eotaxin has changed direction in the multivariable equation. This probably represents a correction for over prediction with the first two variables. This equation predicts correctly predicts presence or absence of HIV in 95.6% of the patients. It predicts HIV− in 127 out of 132 individuals and HIV+ in 70 out of 74 individuals. The predictive equation is

$$p = \frac{1}{1 + {e}^{- z}}$$

where z=5.654+0.003*Eotaxin−0.032*SCF−0.001*PDGF ββ
Values >0.5 predict HIV+

### Examples

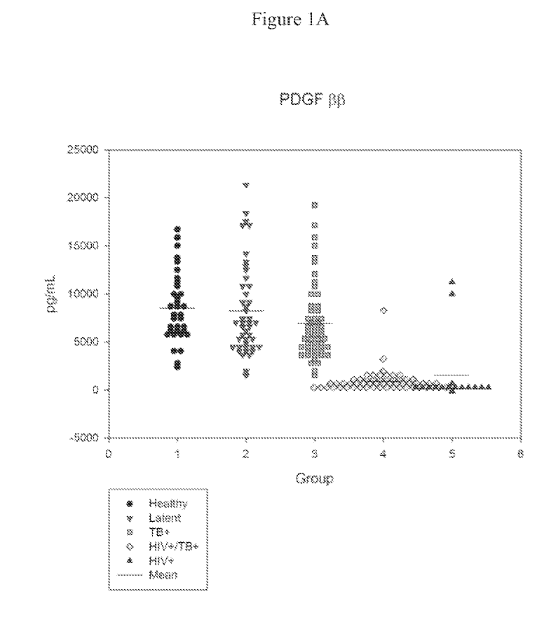

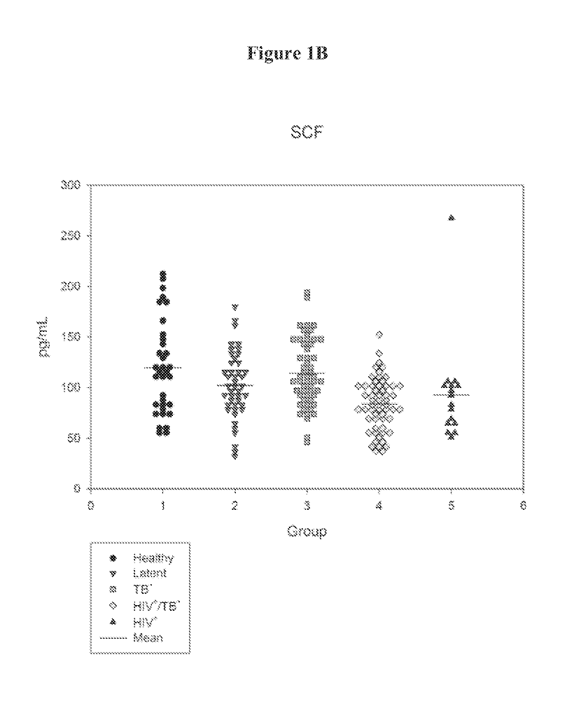

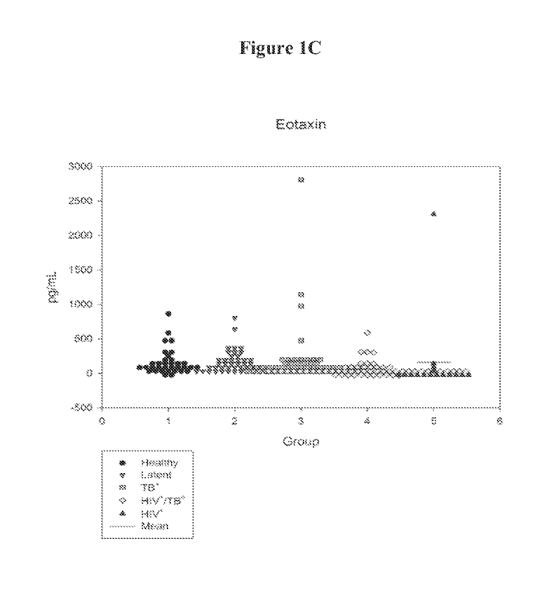

Patient 3 z=5.654+0.003*63.88−0.032*144.91−0.001*6648.05=−5.530 p=1/(1+e^{+5.530})=0.004 predicts HIV− (actual HIV−TB−)
Patient 187 z=5.654+0.003*2.25−0.032*41.37−0.001*1270.15=3.067 p=1/(1+e^{-3.067})=0.956 predicts HIV+(actual HIV+TB+)
   FIGS. 1A-C show HIV frequency plots of cytokines (PDGF ββ, SCF and eotaxin) found in the multivariable logistic regression table.

### TB Logistic Regression

| Cytokine | Odds Ratio | 95% C.I. | p value | OR for δ = 100 |
|---|---|---|---|---|
| IL1-α | 10.205 | 2.644, 39.392 | 0.001 | |
| M-CSF | 1.037 | 1.022, 1.052 | <0.001 | 1.433* |
| TNF-β | 1.896 | 1.319, 2.725 | 0.001 | |
| MCP-3 | 0.992 | 0.987, 0.997 | 0.003 | 0.923* |
| IL-1β | 1.003 | 0.991, 1.016 | 0.617 | |
| G-CSF | 1.011 | 0.988, 1.035 | 0.335 | |
| IL-18 | 1.003 | 1.001, 1.006 | 0.013 | 1.030* |
| GRO-α | 1.011 | 1,006, 1.015 | <0.001 | 1.116* |
| LIF | 1.043 | 1.009, 1.079 | 0.014 | |

Of the 9 cytokines that looked promising in the first step, seven of them are significantly predictive of the presence of HIV using logistic regression. Since TB :s coded as 0=no and 1=yes, Odds Ratios of <1 imply that lower values are associated with presence of TB. An Odds Ratio of >1 would imply that higher values are associated with the presence of TB. The seven significant cytokines were entered into a forward stepwise regression using the Likelihood ratio test to determine which variables entered the equation.

### Multivariable Logistic Regression

| Cytokine | Odds Ratio | 95% C.I. | p value | OR for δ = 100 |
|---|---|---|---|---|
| GRO-α | 1.012 | 1.002, 1.022 | 0.021 | 1.128 |
| MCP-3 | 0.944 | 0.924, 0.964 | <0.001 | 0.560 |
| TNF-β | 1.809 | 1.276, 2.563 | 0.001 | |
| MCSF | 1.068 | 1.024, 1.115 | 0.002 | 1.935 |

### TB Logistic Regression

This equation correctly predicts presence or absence of TB in 90.7% of the patients. It predicts TB− in 42 out of 50 individuals (84%) and TB+ in 105 out of 112 individuals (94%). The predictive equation is:

$$p = \frac{1}{1 + {e}^{- z}}$$

where z=2.146+0.066*MCSF+0.593*TNFβ−0.058*MCP3+0.012GROα
Values >0.5 predict TB+

### Examples

ID3 z=2.146+0.066*154.16+0.593*32.99−0.058*186.36+0.012*329.99=25.035 p=1(1+e^{-25.035})=0.999 predicts TB+(actually HIV+TB+)
ID 137 z=2.146+0.066*40.45+0.593*0.01−0.058*152.2+0.012*192.49=−1.696 p=1/(1+e^{-1.696})=0.155 predicts TB− (actually HIV+TB−)
ID 193 z=2.146+0.066*169.53+0.593*22.03−0.058*313.99+0.012*302.64=11.819 P=1/(1+e^{-11.819})=0.999 predicts TB+ (actually HIV−PPD+TB−)

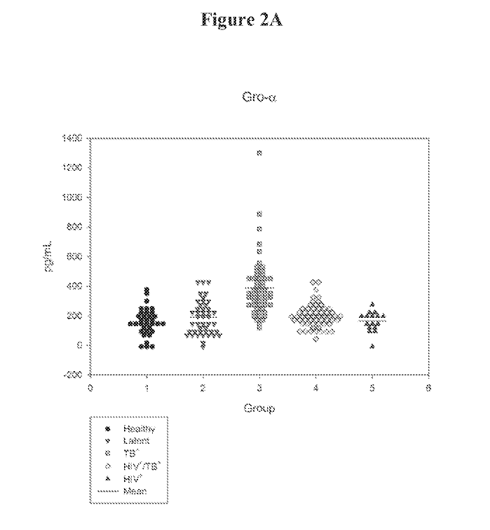

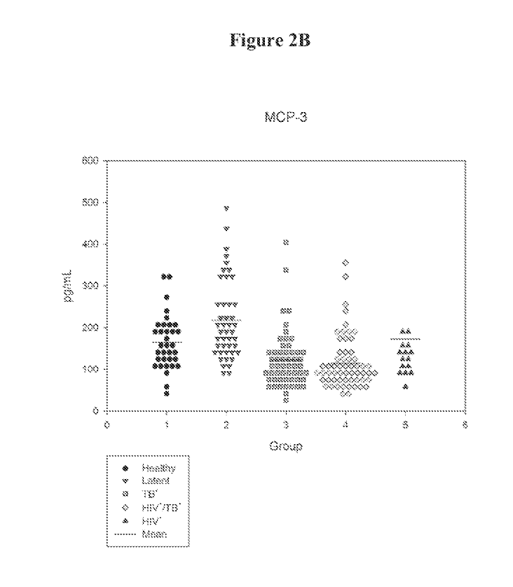

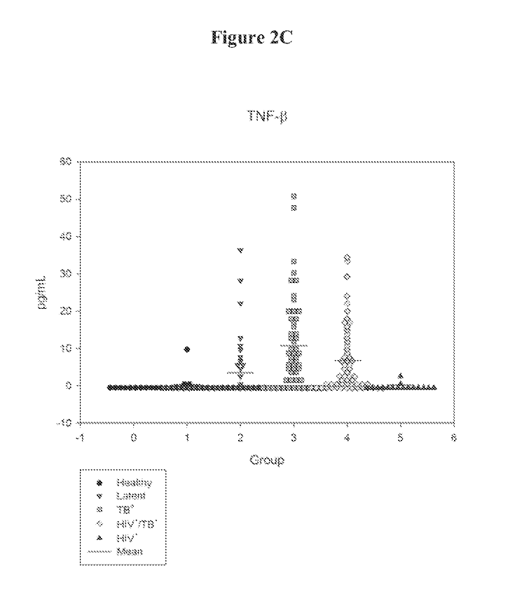

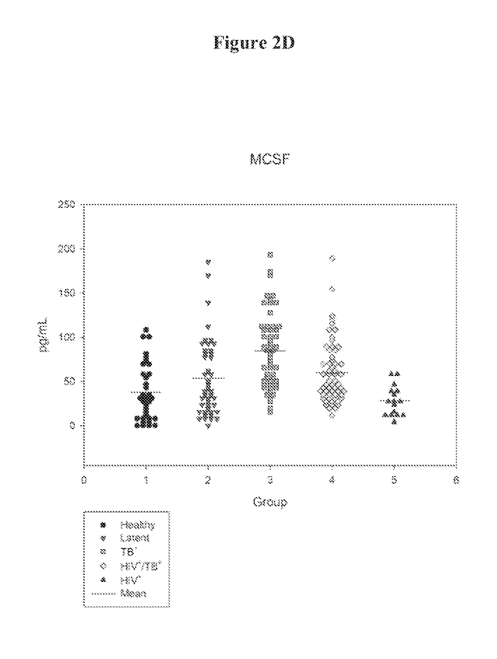

FIGS. 2A-D show TB frequency plots of cytokines (GRO-α, MCP-3, TNF-β and MCSF)) found in the TB multivariable logistic regression table.

PPD+ Vs. All Others Logistic Regression

| Cytokine | Odds Ratio | 95% C.I. | p value |
|---|---|---|---|
| IP-10/100 | 0.980 | 0.960, 1.00 | 0.052 |
| MIP-1α | 0.997 | 0.986, 1.008 | 0.583 |
| IL12 (p40) | 1.000 | 0.999, 1.000 | 0.513 |
| CTACK/100 | 1.096 | 1.012, 1.188 | 0.025 |
| LIF | 0.937 | 0.894, 0.982 | 0.006 |
| IL-3 | 0.998 | 0.995, 1.002 | 0.324 |
| MCP-3 | 1.010 | 1.005, 1.014 | <0.001 |
| TNF-β | 0.963 | 0.918, 1.009 | 0.112 |
| ICAM-1/1000 | 0.838 | 0.777, 0.904 | <0.001 |
| VCAM-1/1000 | 0.831 | 0.764, 0.903 | <0.001 |

Of the 10 cytokines that looked promising in the first step, six of them are significantly predictive of the presence of PPD+ using logistic regression. Since PPD+ is coded as 0=no and 1=yes, Odds Ratios of <1 imply that lower values are associated with presence of PPD+. An Odds Ratio of >1 would imply that higher values are associated with the presence of PPD+. The six significant cytokines were entered into a forward stepwise regression using the Likelihood ratio test to determine which variables entered the equation.

### Multivariable Logistic Regression

| Cytokine | Odds Ratio | 95% C.I. | p value |
|---|---|---|---|
| LIF | 0.946 | 0.908, 0.987 | 0.010 |
| MCP-3 | 1.009 | 1.004, 1.014 | <0.001 |
| CTACK/100 | 1.150 | 1.034, 1.278 | 0.010 |
| ICAM-1/1000 | 0.868 | 0.808, 0.932 | <0.001 |

PPD+ Vs. All Others Logistic Regression

This equation correctly predicts presence or absence of PPD+ in 83.0% of all patients. It predicts PPD+ in 20 out of 44 individuals (45%) and not PPD+ in 151 out of 162 individuals 93%). The predictive equation is

$$p = \frac{1}{1 + {e}^{- z}}$$

where z=−0.611−0.055*LIF+0.009*MCP3+0.001*CTACK−0.141*ICAM/1000
Values >0.5 predict PPD+

### Examples

ID 221 z=−0.611−0.055*0.005+0.009*479.29+0.001*1593.55−0.141*19.14=2.52 p=1/(1+e^{-2.52})=0.93 predicts PPD+ (actual HIV−PPD+TB−)
ID 154 z=−0.611−0.055*0.005+0.009*113.37+0.001*1132.81−0.141*30.08=−2.70 p=1/(1+e^{-2.70})=0.06 predicts not PPD+ (actual HIV−TB−)

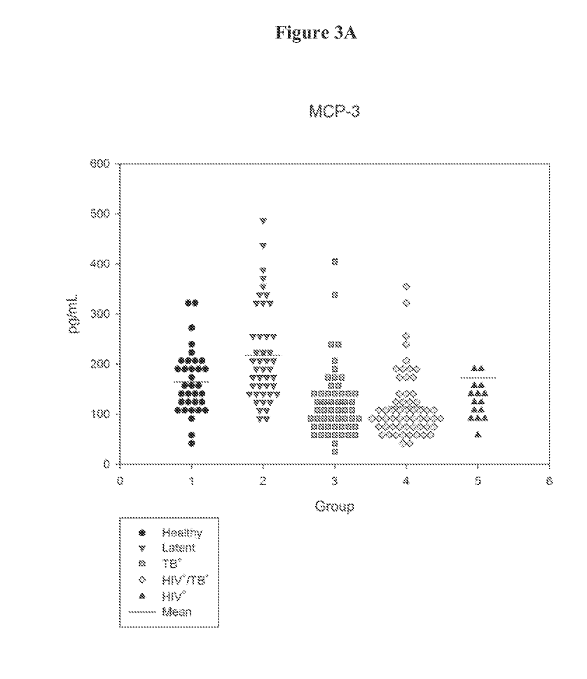

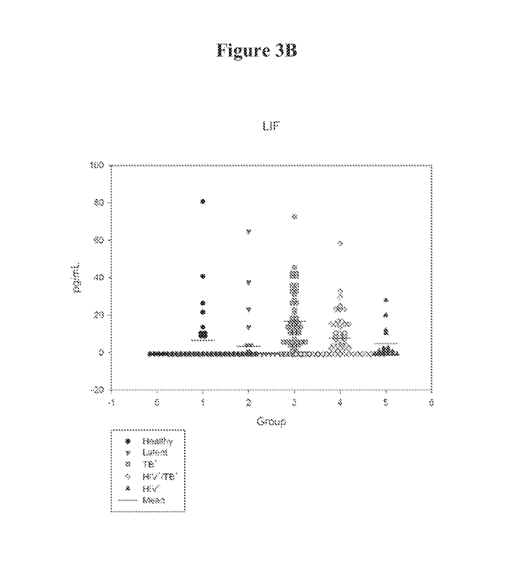

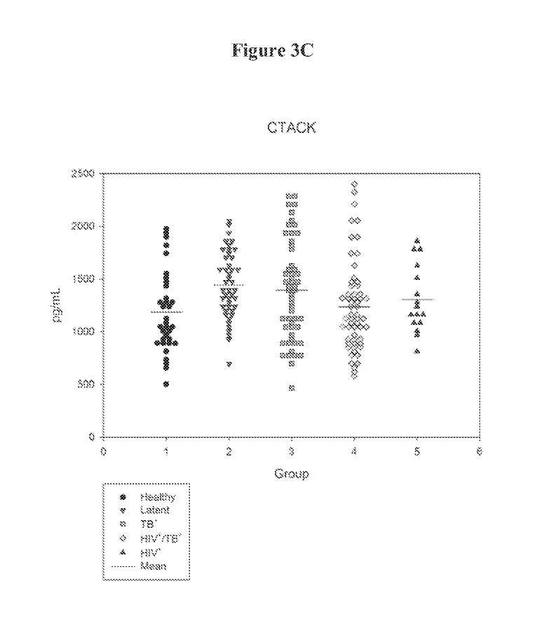

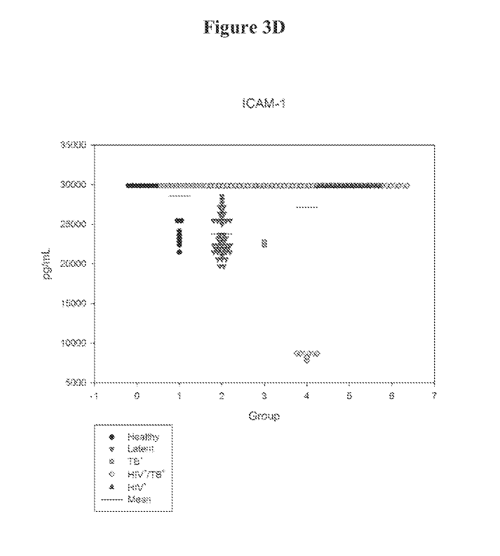

FIGS. 3A-D show PPD+ vs. all others frequency plots of cytokines (MCP-3, LIF, CTACK and ICAM) found in multivariable logistic regression table.

PPD+ Vs. TB− Logistic Regression

| Cytokine | Odds Ratio | 95% C.I. | p value |
|---|---|---|---|
| IP-10/100 | 0.989 | 0.967, 1.011 | 0.313 |
| MIP-1α | 0.996 | 0.984, 1.007 | 0.464 |
| IL12 (p40) | 1.000 | 0.999, 1.000 | 0.201 |
| CTACK/100 | 1.205 | 1.062, 1.369 | 0.004 |
| LIF | 0.983 | 0.948, 1.019 | 0.343 |
| IL-3 | 0.998 | 0.994, 1.002 | 0.265 |
| MCP-3 | 1.005 | 1.000, 1.011 | 0.035 |
| TNF-β | 1.309 | 1.023, 1.675 | 0.032 |
| ICAM-1/1000 | 0.578 | 0.478, 0.698 | <0.001 |
| VCAM-1/1000 | 0.551 | 0.449, 0.676 | <0.001 |

Of the 10 cytokines that looked promising in the first step, five of them are significantly predictive of the presence of PPD+ using logistic regression. Since PPD+ is coded as 0=no and 1=yes, Odds Ratios of <1 imply that lower values are associated with presence of PPD+. An Odds Ratio of >1 would imply that higher values are associated with the presence of PPD+. The five significant cytokines were entered into a forward stepwise regression using the Likelihood ratio test to determine which variables entered the equation.

### Multivariable Logistic Regression

| Cytokine | Odds Ratio | 95% C.I. | p value |
|---|---|---|---|
| ICAM-1/1000 | 0.578 | 0.478, 0.698 | <0.001 |

Only one variable enters the equation. The other variables do not provide additional information. This equation correctly predicts presence or absence of PPD+ in 83.0% of the patients who do not have TB. It predicts PPD+ in 36 out of 44 individuals (82%) and not PPD+ in 42 out of 50 individuals (84%).

PPD+ vs. TB− Logistic Regression
The predictive equation is

$$p = \frac{1}{1 + {e}^{- z}}$$

where z=14.508−0.549*ICAM 1/1000
Values >0.5 predict PPD+

### Examples

ID 221 z=14.508×0.549*19.14=−5.181 p=1/(1+e^{5.181})=0.006 predicts not PPD+ (actual HIV−PPD+TB−)
ID 154 z=14.508−0.549*19.14=−2.006 p=1/(1+e^{+2.006})=0.119 predicts not PPD+ (actual HIV−TB−)

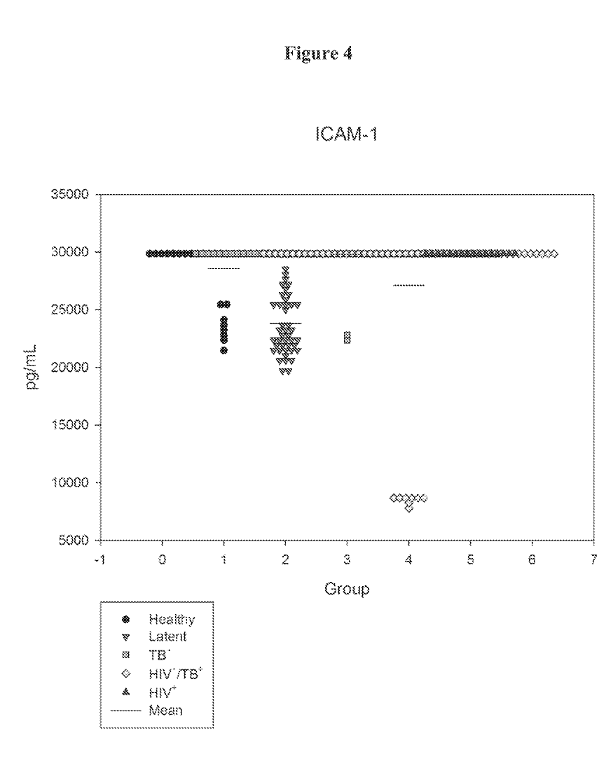

FIG. 4 shows PPD+ vs. TB− frequency plots of cytokines (ICAM-1) found in multivariable logistic regression table.

PPD+ Vs. Healthy Logistic Regression

| Cytokine | Odds Ratio | 95% C.I. | p value |
|---|---|---|---|
| IL 6 | 1.008 | 0.992, 1.024 | 0.316 |
| IL 7 | 0.996 | 0.985, 1.007 | 0.489 |
| GM-CSF | 0.989 | 0.970, 1.009 | 0.291 |
| MCP-1 | 0.977 | 0.956, 0.999 | 0.037 |
| VEGF | 1.001 | 1.000, 1.003 | 0.129 |
| IL-2rα | 1.000 | 0.996, 1.004 | 0.920 |
| CTACK | 1.242 | 1.075, 1.434 | 0.003 |
| ICAM-1/1000 | 0.620 | 0.513, 0.750 | <0.001 |
| MCP-3 | 1.009 | 1.002, 1.015 | 0.011 |
| M-CSF | 1.012 | 0.999, 1.025 | 0.079 |
| TNF-β | 1.278 | 0.982, 1.662 | 0.068 |
| VCAM-1/1000 | 0.601 | 0.488, 0.741 | <0.001 |
| SCGF-β/1000 | 1.007 | 1.001, 1.013 | 0.034 |

Of the 13 cytokines that looked promising in the first step, six of them are significantly predictive of the presence of PPD+ using logistic regression. Since PPD+ is coded as 0=no and 1=yes, Odds Ratios of <1 imply that lower values are associated with presence of PPD+. An Odds Ratio of >1 would imply that higher values are associated with the presence of PPD+. The six significant cytokines were entered into a forward stepwise regression using the Likelihood ratio test to determine which variables entered the equation.

### Multivariable Logistic Regression

| Cytokine | Odds Ratio | 95% C.I. | p value |
|---|---|---|---|
| ICAM-1/1000 | 0.620 | 0.513, 0.750 | <0.001 |

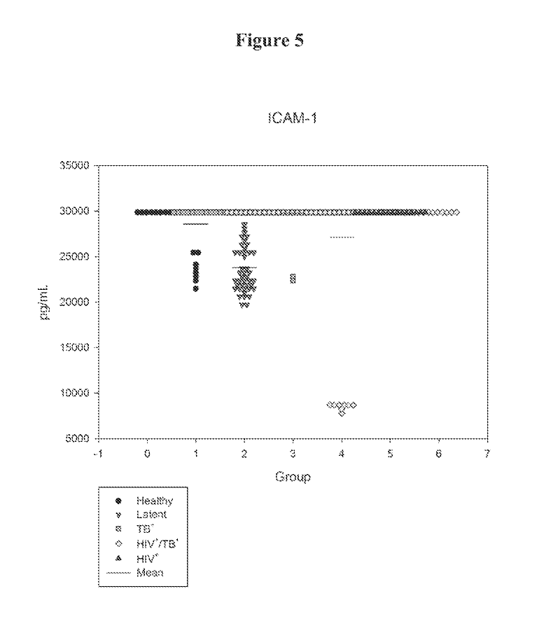

This equation correctly predicts presence or absence of PPD+ in 80.8% of the patients. It predicts PPD+ in 37 out of 44 individuals (84%) and normal in 26 out of 34 individuals (76%). FIG. 5 shows PPD+ vs. healthy frequency plots of cytokines found in multivariable logistic regression table.

1. A method of diagnosing a subject as HIV+ or HIV− comprising: a) measuring the levels of eotaxin, SCF, PDFGbb in a sample from the subject, b) computing a predictive value utilizing the following equation:
$$p = \frac{1}{1 + {e}^{- z}}$$
where z=5.654+0.003*Eotaxin−0.032*SCF−0.001*PDGF ββ
wherein a value >0.5 predicts HIV+, and a value <0.5 predicts HIV−, thus diagnosing the subject as HIV+ or HIV−.

2. A method of diagnosing a subject as TB+ or TB− comprising a) measuring the levels of MCSF, TNFBeta, MCP3, GROalpha in a sample from a subject, b) computing a predictive value utilizing the following equation:
$$p = \frac{1}{1 + {e}^{- z}}$$
where z=2.146+0.066*MCSF+0.593*TNFβ−0.058*MCP3+0.012GROα
wherein a value >0.5 predicts TB+, and a value <0.5 predicts TB−, thus diagnosing the subject as TB+ or TB−.

3. A method of diagnosing a subject as PPD+ or not PPD+ comprising a) measuring the levels of LIF, MCP3, CTACK and ICAM-1 in a sample from a subject, b) computing a predictive value utilizing the following equation:
$$p = \frac{1}{1 + {e}^{- z}}$$
where z=−0.611−0.055*LIF+0.009*MCP3+0.001*CTACK−0.141*ICAM-1/1000
wherein a value >0.5 predicts PPD+, and a value <0.5 predicts not PPD+, thus diagnosing the subject as PPD+ or not PPD+.

4. A method of diagnosing the HIV status and the TB status in a subject comprising:
a) measuring the levels of eotaxin, SCF, PDFGbb in a sample from the subject; and
b) computing a predictive value utilizing the following equation:
$$p = \frac{1}{1 + {e}^{- z}}$$
where z=5.654+0.003*Eotaxin−0.032*SCF−0.001*PDGF ββ
wherein a value >0.5 predicts HIV+, and a value <0.5 predicts HIV−, thus diagnosing the subject as HIV+ or HIV−;
c) measuring the levels of MCSF, TNFBeta, MCP3, GROalpha in a sample from the subject; and
d) computing a predictive value utilizing the following equation:
$$p = \frac{1}{1 + {e}^{- z}}$$
where z=2.146+0.066*MCSF+0.593*TNFβ−0.058*MCP3+0.012GROα
wherein a value >0.5 predicts TB+, and a value <0.5 predicts TB−, thus diagnosing the subject as TB+ or TB−.

5. The method of claim 4, further comprising diagnosing the PPD status of the subject by:
e) measuring the levels of LIF, MCP3, CTACK and ICAM-1 in a sample from the subject; and
f) computing a predictive value utilizing the following equation:
$$p = \frac{1}{1 + {e}^{- z}}$$
where z=−0.611−0.055*LIF+0.009*MCP3+0.001*CTACK−0.141*ICAM/1000,
wherein a value >0.5 predicts PPD+, and a value <0.5 predicts not PPD+, thus diagnosing the subject as PPD+ or not PPD+.

6. The method of claim 4, further comprising diagnosing the PPD status of a subject that is TB− by:
e) measuring the levels of ICAM in a sample from the TB− subject; and
f) computing a predictive value utilizing the following equation:
$$p = \frac{1}{1 + {e}^{- z}}$$
where z=14.508−0.549*ICAM-1/1000
wherein a value >0.5 predicts PPD+, and a value of <0.5 predicts not PPD+, thus diagnosing the TB− subject as PPD+ or not PPD+.

7. The method of claim 1, further comprising treating a subject diagnosed as HIV+ with an effective amount of one or more compounds that decrease HIV infection.

8. The method of claim 2, further comprising treating a subject diagnosed as TB+ with an effective amount of one or compounds that decrease tuberculosis infection.

9. The method of claim 4, further comprising treating a subject diagnosed as TB+/HIV+ with an effective amount of one or more compounds that decrease HIV infection and an effective amount of one or more compounds that decrease tuberculosis infection.

10. The method of claim 5, further comprising treating a subject diagnosed as PPD+ with an effective amount of one or more compounds that prevent tuberculosis infection.
